(19) United States
(12) Patent Application Publication
(10) Pub. No.: US 2008/0051767 A1
(43) Pub. Date: Feb. 28, 2008

(54) CHARACTERIZATION AND MODULATION OF PHYSIOLOGIC RESPONSE USING BAROREFLEX ACTIVATION IN CONJUNCTION WITH DRUG THERAPY

(75) Inventors: Martin Rossing, Coon Rapids, MN (US); Robert S. Kieval, Medina, MN (US); Eric Irwin, Minneapolis, MN (US); Brad Pedersen, Minneapolis, MN (US); Vadim Braginsky, Eagan, MN (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(73) Assignee: CVRx, Inc., Maple Grove, MN (US)

(21) Appl. No.: 11/751,580

(22) Filed: May 21, 2007

Related U.S. Application Data
(60) Provisional application No. 60/802,272, filed on May 19, 2006.

(51) Int. Cl. A61M 5/14

(52) U.S. Cl. 604/891.1; 604/502; 607/3; 607/44

(57) ABSTRACT

A method and device for delivering and monitoring baroreflex and drug therapy to manage hypertension. The method includes providing an implanted an implanted medical device configured to automatically detect drug-related effects on the autonomic nervous system including the steps of measuring a physiologic status of the autonomic nervous system at desired intervals, logging the physiologic status of the autonomic nervous system at desired intervals, monitoring the measured and logged physiologic status of the autonomic nervous system for any changes and correlating the changes to a corresponding drug administration time. The device includes an implanted baroreflex activation device capable of administering one or more hypertension treatment drugs including a controller that activates and adjusts therapy delivery, a baroreflex activation therapy delivery device, a drug therapy delivery device and a device that senses physiologic parameters.

### CROSS-REFERENCES TO RELATED APPLICATIONS

This application claims the benefit of Provisional Patent Application No. 60/802,272, filed May 19, 2006, the disclosure of which is hereby incorporated by reference.

### FIELD OF THE INVENTION

The invention relates generally to medical devices and methods, and more particularly, to utilizing a baroreflex activation device to analyze, and optionally respond to, drug therapy response.

### BACKGROUND OF THE INVENTION

Cardiovascular disease is a major contributor to patient illness and mortality. It also is a primary driver of health care expenditure, costing more than $326 billion each year in the United States. Hypertension, or high blood pressure, is a major cardiovascular disorder that is estimated to affect over 65 million people in the United Sates alone. Of those with hypertension, it is reported that fewer than 30% have their blood pressure under control. Hypertension is a leading cause of heart failure and stroke. It is the primary cause of death for tens of thousands of patients per year and is listed as a primary or contributing cause of death for hundreds of thousands of patients per year in the U.S. Accordingly, hypertension is a serious health problem demanding significant research and development for the treatment thereof.

Hypertension occurs when the body's smaller blood vessels (arterioles) constrict, causing an increase in blood pressure. Because the blood vessels constrict, the heart must work harder to maintain blood flow at the higher pressures. Although the body may tolerate short periods of increased blood pressure, sustained hypertension may eventually result in damage to multiple body organs, including the kidneys, brain, eyes and other tissues, causing a variety of maladies associated therewith. The elevated blood pressure may also damage the lining of the blood vessels, accelerating the process of atherosclerosis and increasing the likelihood that a blood clot may develop. This could lead to a heart attack and/or stroke. Sustained high blood pressure may eventually result in an enlarged and damaged heart (hypertrophy), which may lead to heart failure.

It has been known for decades that the wall of the carotid sinus, a structure at the bifurcation of the common carotid arteries, contains stretch receptors (baroreceptors) that are sensitive to the blood pressure. These receptors send signals via the carotid sinus nerve to the brain, which in turn regulates the cardiovascular system to maintain normal blood pressure (the baroreflex), in part through activation of the sympathetic nervous system. Electrical stimulation of the carotid sinus nerve has previously been proposed to reduce blood pressure and the workload of the heart in the treatment of high blood pressure and angina. For example, U.S. Pat. No. 6,073,048 to Kieval et al. discloses a baroreflex modulation system and method for stimulating the baroreflex based on various cardiovascular and pulmonary parameters. Implantable devices for treating high blood pressure or hypertension by stimulating various nerves and tissue in the body are known and described, for example, in U.S. Pat. No. 3,650,277 (stimulation of carotid sinus nerve), U.S. Pat. No. 5,707,400 (stimulation of vagal nerve), and U.S. Pat. No. 6,522,926 (stimulation of baroreceptors).

Implantable baroreflex activation devices for treating hypertension generally include a pulse generator that stimulates the patient's baroreceptors by applying an electric field to the arterial wall of the carotid sinus artery via an electrode assembly intimately attached to the artery. The pulse generator is controlled by a microprocessor-based controller that may receive feedback from a sensed physiological parameter.

Patients who receive baroreflex activation therapy are likely to also receive drug-based therapy for hypertension and related cardiovascular illnesses. Examples of commonly prescribed medications for control of high blood pressure include the beta blocker esmolol, the calcium channel blocker diltiazem, and the angiotensin converting enzyme (ACE) inhibitor enalapril.

Baroreflex electrotherapy patients may encounter a wide array of problems from a multitude of causes, such as from their underlying condition, from inadvertently taking too much medication, not taking enough medication, taking the wrong medication, or from an adverse interaction between medications. Patient compliance to medication regimens may be poor. Such poor compliance often results in inadequate blood pressure control. A patient's blood pressure control needs may vary from minute to minute, sometimes requiring a raise in blood pressure and other times requiring lowering the patient's blood pressure. Therefore, tight and continuous control of blood pressure is critical for the patient's health. Absent this control, problems can be manifested in many different ways, such as, for example, elevated blood pressure, abnormally low blood pressure, cardiac rhythm anomalies, shortness of breath and changes in normal ventilation, and the like.

Accordingly, an automated, implanted system that delivers electrotherapy and pharmaceutical therapies to monitor and control the patient's blood pressure would be highly beneficial. Implanted baroreflex activation devices have certain physiological monitoring capabilities, which are generally used for configuration and control of the electrotherapy. A need exists for medical care providers to obtain information about drug use or non-use by patients for diagnostic and problem solving purposes. However, because the feasibility of measuring hypertension-related drug effect while administering baroreflex electrotherapy was unknown, utilizing a baroreflex activation device for this purpose has not yet been proposed.

### SUMMARY OF THE INVENTION

According to one aspect of the invention, an implantable baroreflex activation device is configured with a physiological monitoring capability that can distinguish among different activities by the autonomic nervous system, and with a real-time clock. In one embodiment, the device is configured with a data representing a schedule of planned drug administration. The physiological monitoring system measures and logs the patient's autonomic nervous system status for changes or variations, and correlates any observed changes with potentially corresponding drug administration times from the schedule

In a related embodiment, the implanted device communicates with an external data logger or data analyzer. In one such embodiment, the implanted baroreflex activation device carries out signal analysis that enables the device to recognize physiological condition change events that are indicative of drug introduction or cessation of drug therapy, and communicates only the data that is data close in time prior to, and following, such events. In another related embodiment, the implanted device simply outputs raw or partially-analyzed data for further analysis externally.

In another embodiment, the implanted device is pre-configured with drug response profile information. In this type of embodiment, the device compares measured physiological changes, and compares their profile against the pre-configured drug profile to detect certain drug introductions or cessations. This profile information can be specific to the patient, or general to a substantial portion of the population. In the patient-specific embodiment, the pre-configured profiles can be refined for the specific patient over time by entering drug administration information (such as drug type, dosage, administration time, and prescription schedule) into the device via a data input system.

In a related embodiment, the device is preconfigured with, or self-learns the patient's electrotherapy response profile. When analyzing detected physiological changes, the baroreflex activation device can thus take into account any changes in the patient's monitored conditions resulting from the administration or cessation of the electrotherapy. Self-learning is facilitated by the device naturally knowing the time and dosage of the electrotherapy administration.

In one embodiment, the external data collector or analyzer is interfaced with a database in which monitored patient physiological data is logged. This approach enables operations on greater amounts of data representing longer time intervals than can practically be handled by a battery-powered implanted device. In various embodiments, the external data collector/analyzer utilizes known methods of deep data mining, morphology, and regression analysis to identify correlations, patterns, or trends indicative of treatment applications and effectiveness. The external database can also include a larger library of drug effect profiles and known interactions and side effects. This additional information can assist the health care provider in detecting or diagnosing a particular problem condition, or for analyzing, reconstructing, or hypothesizing the root cause of a particular pathology.

According to another aspect of the invention, baroreflex activation is administered in combination with drug therapy to achieve a greater overall reduction in blood pressure. In particular, a surprising synergistic result may be achieved as demonstrated by the results of animal studies described in FIGS. 5a-5d by administering baroreflex electrotherapy concurrently with the drug esmolol. In particular, baroreflex electrotherapy in conjunction with administration of the drug esmolol produced a net reduction of systolic blood pressure that was greater than the sum of the individual reductions achieved by each of the electrotherapy and esmolol by themselves.

In one embodiment, the invention combines baroreflex activation therapy and pharmacological treatment modalities to raise and lower a patient's blood pressure as needed. Drug delivery and baroreflex activation therapy may be delivered together or individually. Use of this combine therapy may allow patient's to take fewer and/or lower doses of drugs, lowering the patient's risk of experiencing side effects associated with such drugs. In a related embodiment, an implanted baroreflex activation device monitors one or more physiological indicators for drug effectiveness, and adjusts the dosage of electrotherapy to achieve or maintain a desired range of conditions in the patient. In another embodiment, the implanted device distinguishes from among observable effects of various drugs to identify the types of one or more drugs administered to the patient. In one such embodiment, the electrotherapy is administered or modulated according to a predefined profile to facilitate identification of drugs administered.

In another aspect of the invention, an implantable baroreflex activation device includes one or more mechanisms for administering selected dosages of one or more drugs including, but not limited to, esmolol, diltiazem, or enalapril. In combination with the above-described arrangements for monitoring the effectiveness of the drug therapy, the baroreflex activation device can adjust the drug and electrotherapy dosages individually to achieve or maintain a desired physiological condition in the patient

In one embodiment, the implanted device maintains information representing amounts of drugs administered or the amounts remaining, as well as the amount of stored energy available for administrating electrotherapy. This type of device can compute, based on the available drug or electrotherapy dosage, an improved treatment regimen to maximize or extend the effective useful life of the device in the patient as a treatment. In a related embodiment, the device characterizes a relative effectiveness of each available treatment as a function of dosage, whether drug type or electrotherapy, as well as for combinations of various drugs, and drugs in combination with electrotherapy, and uses this characterization to find a preferred treatment profile for achieving a desired performance level while extending or maximizing its useful service life.

Various techniques for electrotherapy can be applied according to embodiments of the invention to achieve different types of effects on the patient's autonomic nervous system. For example, in addition to, or in place of stimulating baroreceptors in the carotid sinus artery at the carotid bifurcation, electrostimulation can be applied to the carotid body, to stimulate chemoreceptor cells. Stimulation of the carotid body can produce an effect on the autonomic nervous system that generally opposes the effects resulting from stimulation of the baroreceptors.

Thus, according to one aspect of the invention, autonomic nervous system condition or response is used as part of a control loop capable of both, (a) suppressing sympathetic tone and enhancing parasympathetic tone; and (b) enhancing sympathetic tone and suppressing parasympathetic tone. Embodiments of this aspect include selectively enhancing the desired treatment effectiveness of drug therapy, or counter-acting the drug-induced effects in cases of undesirable drug interactions, over-dose situations, or the accidental ingestion of inappropriate drugs.

To selectively enhance or suppress sympathetic vs. parasympathetic response, an implanted electrostimulation device with multiple electrode assemblies can be utilized, with the first electrode assembly positioned to stimulate baroreceptors a the carotid bifurcation, and the second electrode assembly positioned to stimulate chemoreceptors at the carotid body. In a related embodiment, an single electrode assembly with s plurality of electrode sets includes a first electrode set positioned to stimulate the baroreceptors, while the second electrode set is positioned to stimulate the chemoreceptors

### BRIEF DESCRIPTION OF THE DRAWINGS

FIG. 1 is a diagram illustrating various components of an example baroreceptor/chemoreceptor electrostimulation device that is implantable in a patient. according to one aspect of the invention.

FIG. 2 illustrates one embodiment of a central processing unit (CPU) of the baroreceptor/chemoreceptor electrostimulation device of FIG. 1.

FIG. 3 is a diagram illustrating an exemplary system for assessing events related to drug therapy and baroreflex activation in a patient using an implanted baroreflex activation device.

FIG. 4 is a diagram illustrating an exemplary combined system for assessing, delivering and adjusting drug therapy and baroreflex activation in a patient using an implanted baroreflex activation device/drug delivery system.

FIGS. 5a-5d show the results of animal tests using an embodiment of the invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

One aspect of the invention recognizes that baroreflex activation therapy affects the parasympathetic nervous system in addition to the sympathetic nervous system. Also, the baroreflex activation therapy can affect the sympathetic/parasympathetic balance. According to one embodiment, heart rate variability (HRV) analysis is performed in conjunction with baroreflex activation therapy and drug therapy to measure effectiveness of the electrotherapy and drug therapy on different parts of the autonomic nervous system. For example, the ECG analysis can be utilized to distinguish the sympathetic response to the baroreflex activation or drug therapy from the parasympathetic response.

Techniques are known for measuring the sympathetic and parasympathetic nervous system responses. Beat-to-beat fluctuations which occur around a person's mean heart rate are known as heart rate variability (HRV). The fluctuations from beat-to-beat are attributed, in part, to the nonlinear interaction between the sympathetic and parasympathetic branches of the autonomic nervous system. The sympathetic autonomic and parasympathetic autonomic nervous systems regulate, to some extent, the sinoatrial (SA) node and atrioventricular (AV) node of the heart and, thus, largely influence the control of the heart rate. These two nervous systems operate somewhat reciprocally to effect changes in the heart rate. In this regard, parasympathetic stimulation decreases the firing rate of the pacing cells located in the sinus node of the heart. Sympathetic stimulation, on the other hand, increases this firing rate.

Most clinicians agree that the parasympathetic and sympathetic inputs to the SA node mediate low frequency heart rate fluctuations (i.e., generally below 0.15 Hz), whereas modulation of parasympathetic outflow mediates higher frequency fluctuations. Studies have further shown that a decrease in heart rate variability correlates with a decrease in parasympathetic nervous activity and an accompanied increase in sympathetic nervous activity. See J. Thomas Bigger, et al, “Components of Heart Rate Variability Measured During Healing of Acute Myocardial Infarction” American Journal of Cardiology, Vol. 61 (1988), pp. 208-215. In a healthy, resting heart, for instance, the parasympathetic activity dominates to maintain the heart rate. However, in an unhealthy heart, for example one having heart disease, sympathetic activity may more influence and control the heart rate.

HRV analysis can be performed using time domain and frequency domain measures of variability. Commonly used time domain measures of HRV are concerned with the variability of the interval between the R waves for heart beats with a normal sinus mechanism (NN intervals)Two commonly used measures are the standard deviation of NN intervals (SD), which increases with a reduction in sympathetic tone; and the root mean square of successive differences between adjacent NN intervals (rMSSD), which increases as parasympathetic tone is enhanced.

Frequency domain measures of heart rate variability are typically obtained by performing Fourier analysis, such as fast Fourier transformation (FFT) on sampled sets of ECG recordings, and analyzing changes in the content of certain frequency bins as a function of time. Two peaks are typically present in the FFT of five-minute ECG recordings. High frequency (HF) (0.15-0.40 Hz) peaks reflect modulation of the efferent parasympathetic activity, and low frequency (0.04-0.15 Hz) (LF) peaks reflect modulation of the efferent parasympathetic vagal and efferent sympathetic nervous system. The amplitude of LF or HF power is a measure of autonomic nervous system modulation of sinus node firing, and not a measure of global sympathetic and parasympathetic nervous system tone; however, the LF/HF ratio is used as an index of sympathetic parasympathetic balance. In normal subjects the amplitude of LF power exceeds that of HF; however, during controlled respiration there is a marked increase in HF and a reduction in the LF components and of the LF/HF ratio

Following acute beta-adrenergic blockade with the nonselective betablocker propranolol, which would be expected to result in peripheral sympathoinhibition, there is typically an increase in the HF component and a reduction in the LF component of the FFT of five-minute ECG recordings. This is associated with a reduction in the LF/HF ratio. When blood pressure is reduced by an intravenous infusion of nitroglycerine, or tilt testing, there is typically an increase in the LF component indicating sympathetic activation.

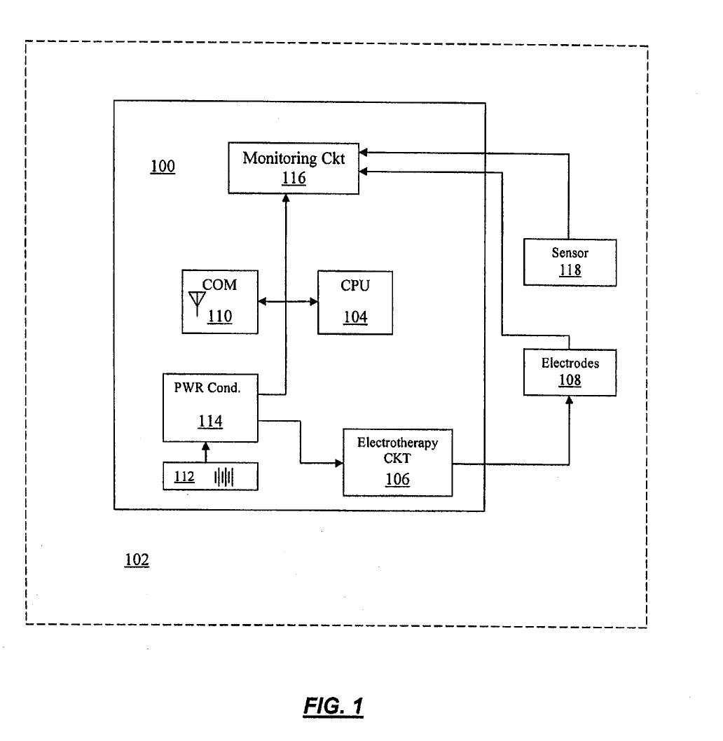

FIG. 1 is a diagram illustrating an example baroreflex activation device 100 that is optionally implantable in a patient 102. Persons of ordinary skill in the art will recognize that the aspects of the invention can be suitably applied to non-implantable, i.e. external baroreflex activation devices. Device 100 includes a central processor unit (CPU) 104, which may include one or more microprocessors or microcontrollers, for example, that is configured to control the operation of the device. CPU 104 is configured to cause the device to administer the electrotherapy via electrotherapy circuit 106 and electrodes 108A communications circuit 110 is interfaced with CPU 104 and is used for communicating information between CPU 104 and equipment external to the patient 102, such as a device programmer (not shown), or external or remote sensors (not shown). Baroreflex activation device 100 also includes a power source such as a battery 112, and power conditioning circuitry 114 for converting the battery power into various power supplies suitable for powering each sub-system of the device. CPU 104 can detect at least one physiologic condition of patient 102 via patient monitoring circuitry 116 and at least one sensor 118.

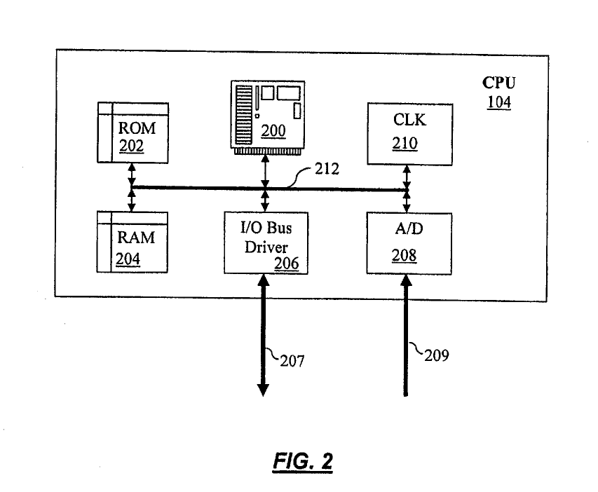

FIG. 2 illustrates one embodiment of CPU 104. CPU 104 includes a microprocessor core 200; read-only memory (ROM) 202 for storing instructions; random access memory (RAM) 204 for use as data gathering, or scratchpad memory during operation; input/output (I/O) bus driving circuitry 206 for transmitting and receiving information via, and controlling the use of, I/O bus 207; analog-to-digital (A/D) converter 208 for converting analog signals received via analog inputs 209 into a digital format for use by microprocessor core 200; and clock 210 for providing a time base for use by microprocessor core 200. In one type of embodiment, CPU 104 has signal processing capability (such as that provided by a DSP core) to perform computations on relatively long sequences of sampled data. An internal CPU interconnect 212 provides an interface between the various CPU components, and can include conventional data exchange hardware, such as a data bus, an address bus, and control lines (not shown).

Referring again to FIG. 1, in a related embodiment, the patient monitoring circuitry 116, or at least a portion of the signal processing circuitry of CPU 104 is situated remotely from device 100 and communicatively coupled with device 100. Similarly, sensor 118 can be remotely situated from patient monitoring circuitry 116 or from device 100

Sensor 118 can take many forms within the spirit of the invention. For example, sensor 118 can include an intravascular or external pressure transducer, arterial pulse detector ultrasonic activity detector, or any suitable device, internal or external to the patient, for sensing or detecting mechanical events or activity of the patient. In other embodiment sensor 118 can be a chemical or optical sensor, such as sensor for measuring a degree of blood oxygenation. Sensor 118 can also include an electrical activity detector, such as one or a set of ECG probes, whether internal or external to the patient. The ECG probes can be of the near-field type that are situated proximally (within 1-2 cm) of the heart or inside the heart, or the far-field type, such as external patches or subcutaneously-implanted electrodes. Sensor 118 can also comprise a set of individual sensors of the same type or of different types.

According to one embodiment of the invention, patient monitoring circuitry 116 operates in cooperation with sensor 118 to collect cardiac activity information for CPU 104. CPU 104 processes this cardiac activity information to produce a characterization of the patient's condition being monitored. In one embodiment, monitoring circuitry 116 and sensor 118 collect cardiac rhythm information, such as the time difference between R-wave peaks, or the period or frequency of detected arterial pulses or heart beats. CPU 104 analyzes this cardiac rhythm information according to heart rate variability (HRV) analysis techniques, such as those described above, and produces an evaluated score or some other quantitative assessment of the HRV analysis

In one embodiment, a combination of electrical cardiac rhythm sensing is correlated to detected arterial pulses. This type of scheme can provide cardiac electrophysiology information in relation to heart contractility information. Processor 104 can use this information to make additional inferences or diagnoses of the patient's condition. For example, differences between the HRV as computed based on by an ECG type measurement, versus the HRV as computed by a pulse detection arrangement may provide important diagnostic insight in to a systemic cause of an observable disease.

In a related embodiment, processor 104 conducts HRV analysis so as to distinguish the effectiveness of the electrotherapy as affecting the sympathetic nervous system response, or as affecting the parasympathetic nervous system response. This degree of analytical insight can be instituted in concert with other, symptomatic- or sign-oriented, physiological sensing such as blood pressure, pulse oximetry, and the like. Processor 104 can further process these various physiological measurements or characterizations to synthesize the different types of information into a comprehensive patient condition assessment. Analytical methods can include regression analysis, morphology, and other computational techniques that are known in the art

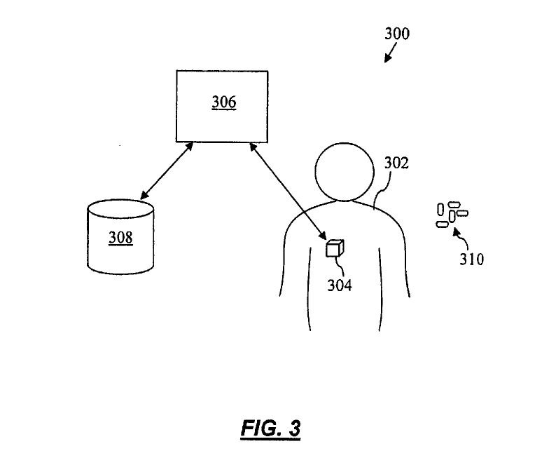

FIG. 3 is a diagram illustrating an exemplary system 300 for assessing events related to drug therapy and baroreflex activation in patient 302 using an implanted baroreflex activation device 304. Implanted device 304 detects various autonomic nervous system indicia, such as by one or a combination of the patient monitoring mechanisms described above. In one embodiment, the implanted device 304 wirelessly transmits (such as by RF transmission) monitored data to external data collector/analyzer 306. External data collector/analyzer 306 is, in turn, interfaced with a database 308, which logs the patient data and other related information. Database 308 can also include a drug introduction timeframe, correlation rules for recognizing certain types of changes to the patient's baseline measurements, drug effect measurement information, and baroreflex activation measurement information. In one embodiment, database 308 further includes various algorithms for conducting different analyses of the monitored patient physiology information

Data collector/analyzer 306 processes the monitored patient data and analyzes the data according to any of the above-described techniques to recognize certain physiological effects resulting from administration or cessation of drug 310, or of administration or cessation of electrotherapy. The analysis program can assign a score to various identified events based on their respective degree of correlation to drug introduction, for example. In one embodiment, data collector/analyzer 306 generates a report for a health care provider that summarizes or highlights certain events having a relatively higher score. In a related embodiment, the report includes deductions, inferences, or conclusions presented for the physician's consideration, including the bases from which the deductions, inferences or conclusions are derived. The report can include deductions, inferences, or conclusions about patient compliance with prescriptions and about possible drug interactions based on measured physiological indicia.

In a related embodiment, implanted device 304 performs at least some of the data analysis to recognize either a drug-related physiological change, or to recognize an increased likelihood of a drug-related physiological effect, and adjusts the electrotherapy dosage to maintain the patient's autonomic nervous system indications within a predefined range.

In one embodiment of the invention, sensors may enable the system to operate in a closed loop fashion by providing physiologic information to determine when, at what amount, and at what rate the baroreflex activation therapy and/or drug therapy should be delivered and a controller to adjust the therapy based on the sensed information. Sensors may provide frequent and even continuous monitoring of physiologic parameters to optimally control the patient's blood pressure improving patient outcomes.

The drug delivery may be systemic or may be directed to specific target organs. One or more delivery lines may be used to deliver the drug therapy. Drug delivery may be accomplished via any route, including oral and/or parenteral. According to one aspect of the invention, baroreflex activation therapy may be modified subsequent to the delivery of drug therapy and/or baroreflex activation therapy may be modified upon sensing the effects of the drug therapy. The baroreflex activation therapy may activate more than one nerve and/or nerve pathway to raise and/or lower blood pressure. Conversely, the drug therapy may be modified following baroreflex activation therapy and/or upon sensing the effects of baroreflex activation therapy.

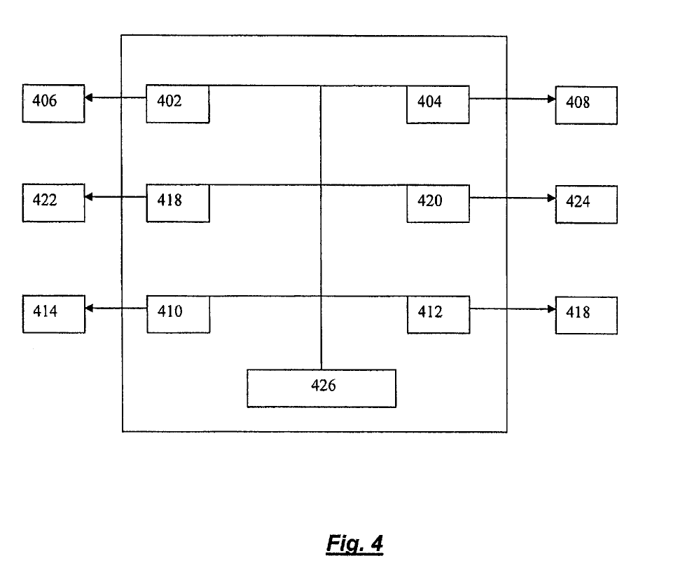

A diagram of a combined baroreflex activation and drug therapy device is shown in FIG. 4. According to this example embodiment, the combined device includes two baroreflex activation devices 402 and 404 each operably connected to respective baroreflex therapy leads 406 and 408. The combined device may also include two drug therapy devices 410 and 412 each operably connected to respective drug delivery ports 414 and 416. In addition, the combine device may include two sensors 418 and 420 each operably connected to respective sensor leads 422 and 424. In this example embodiment, a controller 426 may be hardwired or in wireless communication with baroreflex devices 402 and 404, sensors 418 and 420 and drug therapy devices 410 and 412. Devices 402 and 404 may be combined in a single housing with drug therapies 406 and 408, or separate housings may be used. If separate housings are used, the devices may communicate wirelessly or they may be hardwired.

In one embodiment, sensors 418 and 420 may monitor physiologic information from the patient and transmit this information to controller 426. Controller 426 may include data collector/analyzer 428 that processes the monitored patient data and analyzes the data according to any of the above-described techniques to recognize certain physiological effects resulting from administration or cessation of drug therapy or of administration or cessation of electrotherapy. Controller 426 may then adjust the drug and/or baroreflex activation therapy to raise or lower the patient's blood pressure as needed.

In an embodiment, the system may also include remote communication and programming capabilities such that the treating physician may monitor and adjust treatment as desired. In a related embodiment, the system may include a disposable pill bottle that may transmit a signal to the system when the bottle is opened and a pill is dispensed. This signal may be received by the system informing controller 426 that a drug has been delivered orally. In another embodiment, the system may include additional treatment modalities such as for example, cardiac pacing, defibrillation, other neurostimulation, other drug delivery and the like.

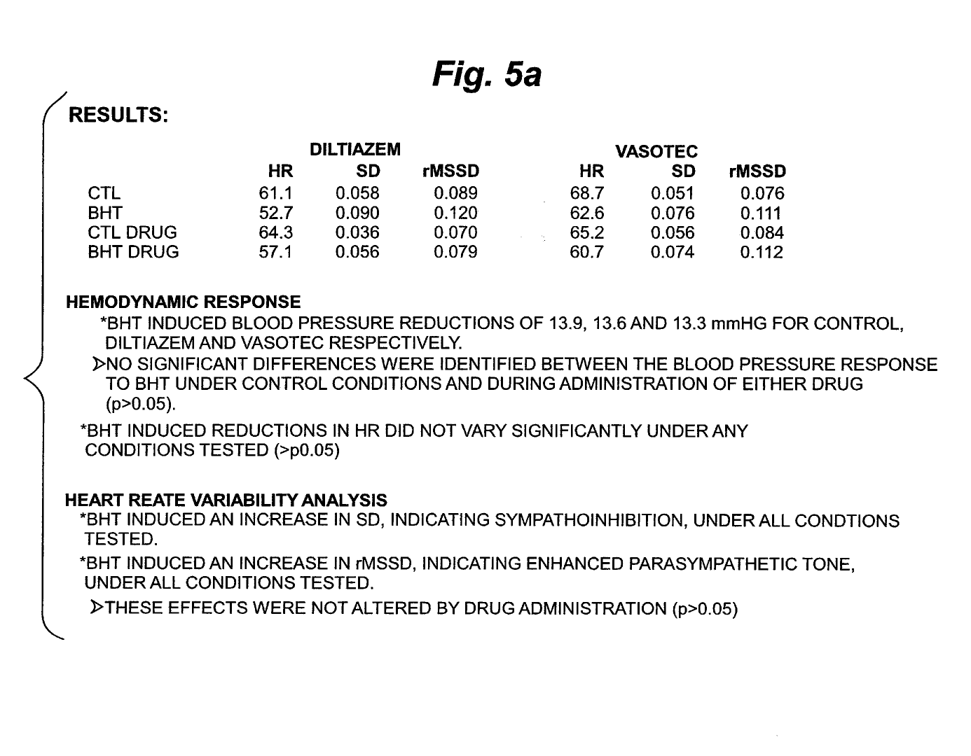

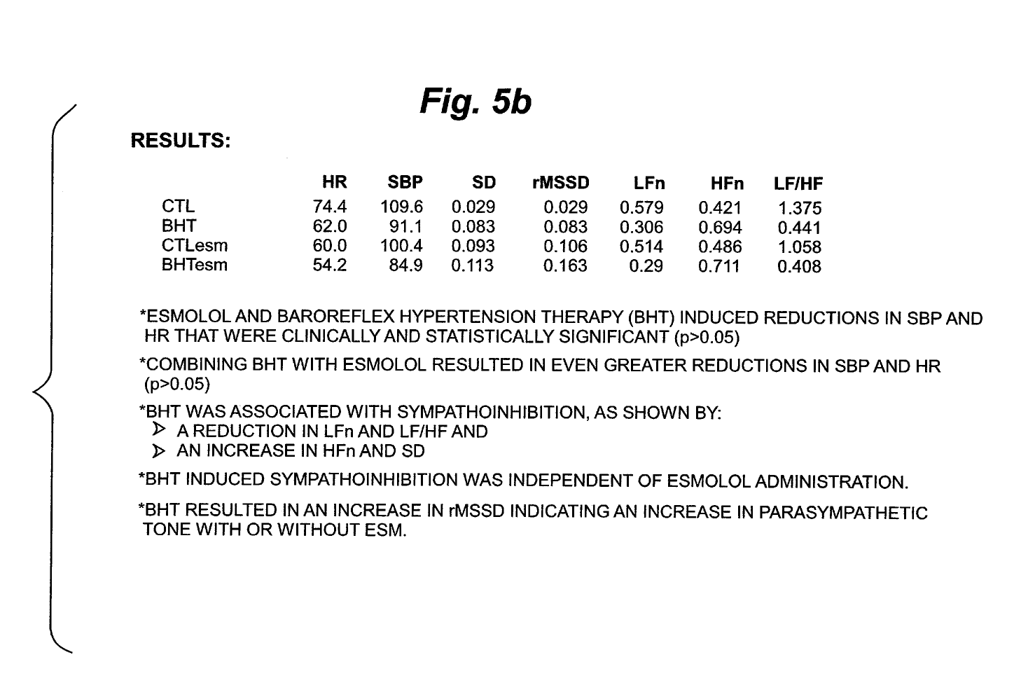

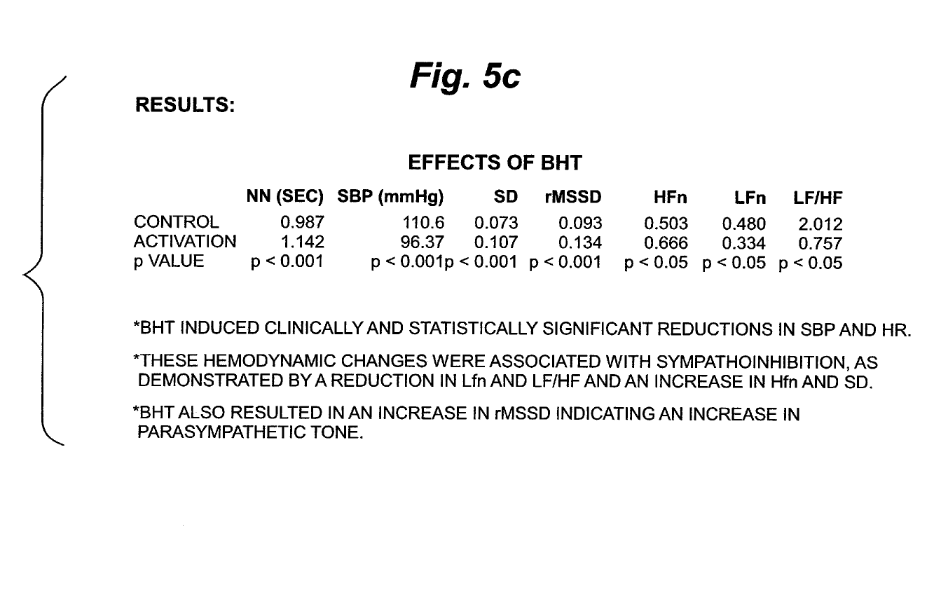

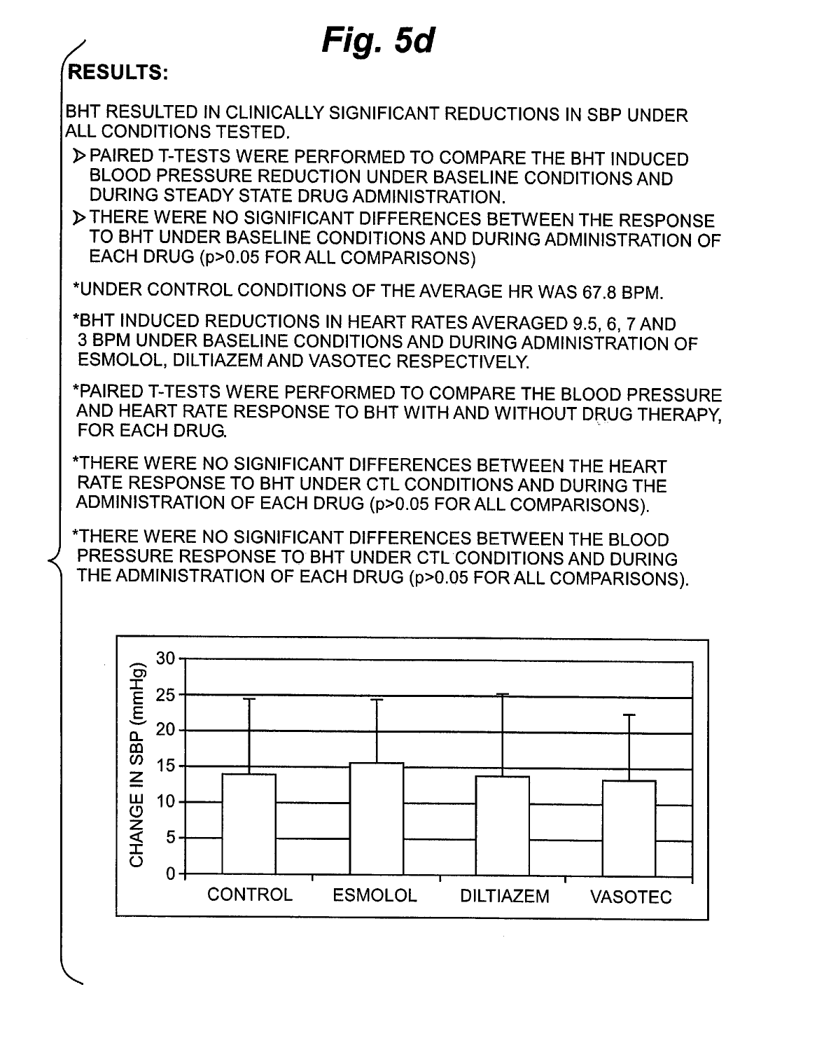

A surprising synergistic result may be achieved as demonstrated by the results of animal studies described in FIGS. 5a-5d by administering baroreflex electrotherapy concurrently with the drug esmolol. In particular, baroreflex electrotherapy in conjunction with administration of the drug esmolol produced a net reduction of systolic blood pressure that was greater than the sum of the individual reductions achieved by each of the electrotherapy and esmolol by themselves. FIGS. 5a-5d show the results of animal testing using various applications of certain aspects of the present invention related to administering drug therapy in conjunction with electrotherapy, and characterizing performance of the drug therapy and baroreflex activation therapy, the disclosure of which is incorporated as part of this application. FIG. 5a shows that sympathoinhibition induced by baroreflex activation therapy is preserved during the administration of antihypertensive medicines in canines. FIG. 5b shows that baroreflex activation therapy enhances the antihypertensive effects of Beta-adrenergic blockade and induces central sympathoinhibition in canines. FIG. 5c shows that a reduction of sympathetic tone and enhanced parasympathetic tone with baroreflex activation therapy contributes to the reduction of blood pressure observed with baroreflex activation therapy. FIG. 5d shows that the hemodynamic response to baroreflex activation therapy is maintained during the administration of antihypertensive medications in normotensive canines.

Additional disclosure material that exemplifies at least a portion of the other features and functionality of the range of embodiments within the spirit and scope of the present invention can be found in Published U.S. Patent Application No. 2005/0154418 to Kieval et al., Published U.S. Patent Application No. 2005/0251212 to Kieval et al., and Published U.S. Patent Application No. 2006/0293712 to Kieval et al., the disclosures of which are hereby incorporated by reference in their entireties. Additional disclosure material relating to vascular anatomy and the cardiovascular system as it pertains to the present invention can be found in U.S. Pat. No. 6,522,926 to Kieval et al., the disclosure of which is hereby incorporated by reference.

Although the description of the present invention is focused on baroreflex activation therapies based on electrical stimulation of the baroreflex system, other forms of baroreflex activation are fully within the spirit and scope of the invention. For example, various forms of mechanical baroreflex activation and chemical baroreflex activation are applicable to the embodiments disclosed herein. Additional disclosure relating to mechanical and chemical forms of baroreflex therapy can be in U.S. Pat. No. 6,522,926, previously incorporated by reference.

Various modifications to the invention may be apparent to one of skill in the art upon reading this disclosure. For example, persons of ordinary skill in the relevant art will recognize that the various features described for the different embodiments of the invention can be suitably combined, un-combined, and re-combined with other features, alone, or in different combinations, within the spirit of the invention. Likewise, the various features described above should all be regarded as example embodiments, rather than limitations to the scope or spirit of the invention. Therefore, the above is not contemplated to limit the scope of the present invention.

For purposes of interpreting the claims for the present invention, it is expressly intended that the provisions of Section 112, sixth paragraph of 35 U.S.C. are not to be invoked unless the specific terms “means for” or “step for” are recited in a claim.

What is claimed is:

1. A method of automatically detecting drug-related effects on the autonomic nervous system comprising:
providing an implanted medical device configured to automatically detect drug-related effects on the autonomic nervous system including the steps of:
measuring a physiologic status of the autonomic nervous system at desired intervals;
logging the physiologic status of the autonomic nervous system at desired intervals;
monitoring the measured and logged physiologic status of the autonomic nervous system for any changes; and
correlating the changes to a corresponding drug administration time.

2. A baroreflex activation device comprising:
a monitor configured to monitor an autonomic nervous system condition; and
a central processing unit configured to correlate the condition to a known effect associated with an introduction or cessation of a hypertension treatment drug.

3. A system for automatically monitoring and detecting drug-related physiology changes comprising:
an implantable device including:
a sensor adapted to sense physiologic changes at desired intervals;
a central processing unit adapted to analyze the physiologic changes and correlate the changes to a drug delivery time.

4. The system of claim 3 further including a baroreflex activation device.

5. The system of claim 3 further including a drug delivery device.

6. A method of treating hypertension by administrating drug therapy in conjunction with baroreflex activation therapy comprising:
implanting a combined drug therapy and baroreflex activation therapy device in a patient;
delivering drug therapy and baroreflex activation therapy to a patient at desired intervals.

7. The method of claim 6 further including measuring physiologic parameters after at least one of the baroreflex and/or drug therapy delivery.

8. The method of claim 7 further including adjusting at least one of the drug and/or baroreflex activation therapy based on the measured physiologic parameters.

9. The method of claim 6 including measuring physiologic parameters at desired intervals.

10. The method of claim 6 further including correlating the measured physiologic parameters to a corresponding delivery time of at least one of the drug and/or baroreflex activation therapy.

11. An implanted baroreflex activation device with means for administering one or more hypertension treatment drugs comprising:
a controller means for activating and adjusting therapy delivery;
a means for delivering baroreflex activation therapy;
a means for delivering drug therapy and
a means for sensing physiologic parameters.

12. A method, comprising:
providing an implantable baroreflex activation device;
providing a sensing arrangement;
providing an implantable drug delivery device;
providing a controller operably communicable with the baroreflex activation device, the sensing arrangement and the drug delivery device; and
providing instructions for operating the device, comprising:
implanting a drug therapy device and a baroreflex activation therapy device in a patient and
delivering drug therapy and baroreflex activation therapy to a patient at desired intervals.

13. A baroreflex therapy system, comprising:
an implantable baroreflex activation device;
a sensing arrangement;
an implantable drug delivery device;
a controller in operable communication with the baroreflex activation device, the sensing arrangement and the drug delivery device; and
instructions recorded on a tangible medium for operating the system, comprising:
measuring a physiologic status of the autonomic nervous system at desired intervals;
logging the physiologic status of the autonomic nervous system at desired intervals;
monitoring the measured and logged physiologic status of the autonomic nervous system for any changes; and
correlating the changes to a corresponding drug administration time.

14. A method, comprising:
providing an implantable baroreflex activation device;
providing an implantable drug delivery device;
providing a sensing arrangement;
providing a controller operably communicable with the baroreflex activation device, the drug delivery device and the sensing arrangement; and
providing instructions for operating the device, comprising:
measuring cardiac electrical activity of a patient with the sensing arrangement to generate cardiac electrical activity data;
communicating the cardiac electrical activity data to the controller;
performing heart rate variability analysis with the controller based on the cardiac electrical activity data; and
providing an indication of the results of the heart rate variability analysis to determine an effect on the autonomic nervous system of a patient, upon which a determination may be made to adjust at least one of the baroreflex therapy and drug therapy to be delivered by at least one of the implantable baroreflex activation device and the drug delivery device.

15. A baroreflex therapy system, comprising:
an implantable baroreflex activation device;
a sensing arrangement;
an implantable drug delivery device;
a controller in operable communication with the baroreflex activation device, the drug delivery device and the sensing arrangement; and
instructions recorded on a tangible medium for operating the device, comprising:
measuring cardiac electrical activity of a patient with the sensing arrangement to generate cardiac electrical activity data;
communicating the cardiac electrical activity data to the controller;
performing heart rate variability analysis with the controller based on the cardiac electrical activity data; and
providing an indication of the heart rate variability analysis upon which a determination may be made to adjust at least one of a baroreflex therapy and drug therapy to be delivered by at least one of the implantable baroreflex activation device and the drug delivery device.
